(19) United States
(12) Patent Application Publication
(10) Pub. No.: US 2008/0008759 A1
(43) Pub. Date: Jan. 10, 2008

(54) Method of improving wound healing

(75) Inventor: Robert Ibsen, Santa Maria, CA (US)

Correspondence Address: CROWELL & MORING LLP;INTELLECTUAL PROPERTY GROUP, P.O. BOX 14300, WASHINGTON, DC, 20044-4300, US

(73) Assignee: Den-Mat Corporation, Santa Maria, CA (US)

(21) Appl. No.: 11/806,235

(22) Filed: May 30, 2007

Related U.S. Application Data
(60) Provisional application No. 60/808,958, filed on May 30, 2006.

(51) Int. Cl. A61K 31/74; A61K 9/00; A61P 17/02

(52) U.S. Cl. 424/486; 424/487; 424/78.06

(57) ABSTRACT

The present invention relates to method of wound healing. More particularly, the present invention is directed to of treating wounds using ionomer resins. The invention involves treating the injured tissue with biocompatible crosslinked resins that optionally contain leachable healing and anti-biotic agents.

### FIELD OF THE INVENTION

The present invention relates to wound healing. More particularly, the present invention is directed to method and compositions for improving wound healing.

### BACKGROUND OF THE INVENTION

The invention relates to methods and compositions for aiding tissue repair by promoting the growth of skin tissue with which they are in contact. The invention involves treating the injured tissue with biocompatible crosslinked resins that optionally contain leachable healing and anti-biotic agents.

Early materials used to treat skin wounds included medicated and unmedicated cotton wools, gauzes, tows, and lints; gauze and cotton tissues, bandages, jaconet, oiled silk and emplastrums. From 1960 on, a new generation of products was developed based on the realization that the control of micro-environments was necessary if wound healing was to progress to the optimum degree.

Advances in the development of synthetic polymers produced the most radical changes in wound care dressings as factors such as water vapor, oxygen permeability, bacterial impermeability, and selective absorption could be incorporated into new formulations along with specific requirements such as conformability, non-adherence, and adhesiveness. This family of polymeric products included polymeric foams, polymeric films, particulate and fibrous polymers, hydrogels and hydrocolloids.

Hydrogels are three dimensional cross-linked networks of hydrophilic polymers that are prepared from materials such as gelatin, polysaccharides, cross-linked polyacrylamide polymers, polyelectrolyte complexes, and polymers or copolymers derived from methacrylate esters. These interact with aqueous solutions by swelling to an equilibrium value and retaining a significant proportion of water within their structure. They are insoluble in water.

In contrast to the single polymer hydrogels described, the products designated as hydrocolloids are complex formulations that contain not only colloids but elastomeric and adhesive components. Hydrocollids have an adhesive formulation that gives an initial adhesion higher than some surgical adhesive tapes. After application, the absorption of transepidermal water vapor will modify the adhesive flow to maintain a high tack and adhesive performance throughout the period of use.

A common problem in the management of both acute and chronic wounds is the maintenance of an optimal level of moisture over the wound bed during heavy exudate drainage. This is usually, but not always, an early stage of healing. Most moist wound dressing technologies such as thin films, hydrocolloid dressings and hydrogels are typically overwhelmed by the accumulated exudate moisture during this heavy drainage phase. Management of moisture during heavy exudate drainage often necessitates the use of gauze or sponge packings that wick away excess moisture from the wound bed, thin film coverings that trap exudate fluid over the wound bed, or calcium alginate dressings that chemically bind exudate moisture due to the hydroscopic properties of the seaweed extract.

Known hydrocolloid dressings are subject to a number of drawbacks. The major disadvantages of these dressings include the potential to disintegrate in the presence of excess fluid at the wound site, and minimal, virtually negligible, control over water loss from the wound. This latter disadvantage is particularly important, as excess water loss from a wound will cause an increase in heat loss from the body as a whole, potentially leading to hypermetabolism. In addition, hydrocolloid dressings require frequent dressing changes.

There has also been proposed the use of a biocompatible wound dressing based on fibrin. One mechanism for hemostasis, i.e., prevention of blood loss, of a mammal is the formation of a blood clot. Clot formation in humans, i.e., blood coagulation, occurs by means of a complex cascade of reactions with the final steps being the conversion of fibrinogen—a monomer—by thrombin, calcium ions and activated factor XIII to form ultimately crosslinked fibrin II polymer, which is the fibrin clot.

The formation of crosslinked fibrin II polymer proceeds by the fibrinogen being converted by thrombin to fibrin I monomer, which spontaneously polymerizes to form fibrin I polymer, which is sometimes referred to as soluble fibrin I because by treatment by appropriate chemical means the fibrin I polymer can be reconverted to fibrin I monomer. The fibrin I polymer is then converted by thrombin to fibrin II polymer, which is sometimes referred to as soluble fibrin II because by treatment by appropriate chemical means the fibrin II polymer can be converted to fibrin II monomer. The fibrin II polymer, under the influence of factor XIIIa—known as activated factor XIII—is then crosslinked to form crosslinked fibrin II, which is the fibrin clot. Factor XIII is activated by thrombin in the presence of calcium ions. Cross-linked fibrin II is sometimes referred to as insoluble fibrin II because it cannot be converted to fibrin II monomer.

Fibrinogen represents about 2 to 4 grams/liter of the blood plasma protein. Fibrinogen is a monomer that consists of three pairs of disulfide-linked polypeptide chains designated (a)₂, (β)₂, (γ)₂ “A” and “B” represent the two small aminoterminal peptides, known as fibrinopeptide A and fibrinopeptide B, respectively. The cleavage of fibrinopeptides A from fibrinogen in the transformation of fibrinogen by thrombin results in the fibrin I compound and the subsequent cleavage of fibrinopeptides B results in the fibrin II compound. Such cleavage of fibrinopeptides A and B reduces the molecular weight of fibrinogen by an extremely small amount, about 6,000 out of 340,000 daltons, but exposes the polymerization sites.

A fibrin sealant is a biological adhesive whose effect imitates the final stages of coagulation, thereby resulting in a fibrin clot. Conventional fibrin sealants consist of concentrated human fibrinogen, bovine aprotinin and factor XIII, as the first component and bovine thrombin and calcium chloride as the second component. Application is generally carried out with a double-barrelled syringe, which permits simultaneous application of both components to the site where one wants to form the fibrin clot. Aprotinin is a fibrinolytic inhibitor added to promote stability of fibrin sealants. U.S. Pat. No. 6,310,267, issued to Rapp, discloses a fibrin based wound covering with a biodegradable carrier support.

Wound dressings have also been combined with a biodegradable carrier material. Common carriers include natural or chemically modified collagen, keratin, gelatin, carbohydrates or cellulose derivatives. Synthetic, biodegradable polymer carriers have also been proposed. These include polyhydroxycarboxylic acids, polyesters, polycyanoacrylates, polyamino acids, polyalcohols and silicones. These carrier materials are commonly employed as a web or as a fabric.

Collagen carriers suffer from numerous deficiencies. Collagen films do not readily conform to varied wound shapes. Furthermore, some collagen wound dressings have poor fluid absorption properties and undesirably enhance the pooling of wound fluids.

Wound dressings have also been combined with numerous pharmacological and/or antibiotic compositions. Examples of such compositions include, but not are not limited to, antifungal compositions, anti-viral compositions, antibacterial compositions, and antiparasitic compositions Examples of antimicrobial compositions that can be used in the present invention include, but are not limited to, isoniazid, ethambutol, clofazimine, rifabutin, fluoroquinolones, pyrazinamide, streptomycin, ofloxacin, ganciclovir, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, erythromycin, ciprofloxacin, clindamycin, lincomycin, acyclovir, trifluorouridine, pentamidine, atovaquone, paromomycin, diclazaril, acyclovir, trifluorouridine, foscarnet, penicillin, gentamicin and sparfloxacin.

Thus far, current compositions and methods of enhancing the natural healing process of wounds, e.g., current wound dressings, have been inadequate in many instances. The inventors of the instant invention have unexpectedly discovered that uncured ionomer-resins have surprising curative properties when applied directly to wounds.

Geristore®, sold by Den-Mat Corporation, Santa Maria, Calif., is promoted for certain uses in dentistry. U.S. Pat. Nos. 4,738,722, 5,334,625, 5,151,453, and 5,876,743, incorporated herein by reference, describe Geristore® and its uses. Geristore® is a small particle composite that contains fluoride, is radiopaque and hydrophilic. It has low-cure shrinkage, low coefficient of thermal expansion and high strength. It aggressively bonds by chemical coupling to dentin, enamel, composites used in dentistry, porcelain and metal, such as stainless steel. It is a paste/paste formulation that is easy to mix. It is capable of rapid cure by exposure to room temperature and for more rapid cure, by exposure to light. In addition, though it contains a fluoride, which could be toxic when ingested in large dosages, it is biocompatible and safe to use within a human or other animal when applied topically.

U.S. Pat. No. 5,876,743 describes a method for aiding in the healing of an open wound or an exposed wound (such as a subcutaneous, penetrating (including a traumatopneic wound), perforating, or tangential wound) which comprises superimposing a cured layer of primary coating with fluoride onto the wound until such time as the wound is closed as a result of the healing process. U.S. Pat. No. 5,876,743 does not disclose the application of uncured ionomer-resins directly to wounds to facilitate healing.

Body tissues are oftentimes subjected to undesirable afflictions such as wounding, irritation, decay or damage of bone or soft tissue. Irritation can be reflected in inflammation, decay can involve erosion and/or decomposition of tissue, and damage can be a wound or fracture. The present invention involves topically treating mammalian, preferably human and domestic animal, wounds with Geristore® to improve the natural healing of such afflictions.

### SUMMARY OF THE INVENTION

One embodiment of the invention encompasses a process for enhancing the normal healing processes of a wound by treating the wound with an ionomer-resin.

Another embodiment of the invention encompasses a process for enhancing the normal healing processes of a wound by topically treating the wound with an ionomer-resin further comprising a pharmacological, healing or antibiotic agent.

### BRIEF DESCRIPTION OF THE FIGURES

FIG. 1. Photo shows an infected wound taken at 36 hours after injury, 12 hours after treatment with Geristore®.

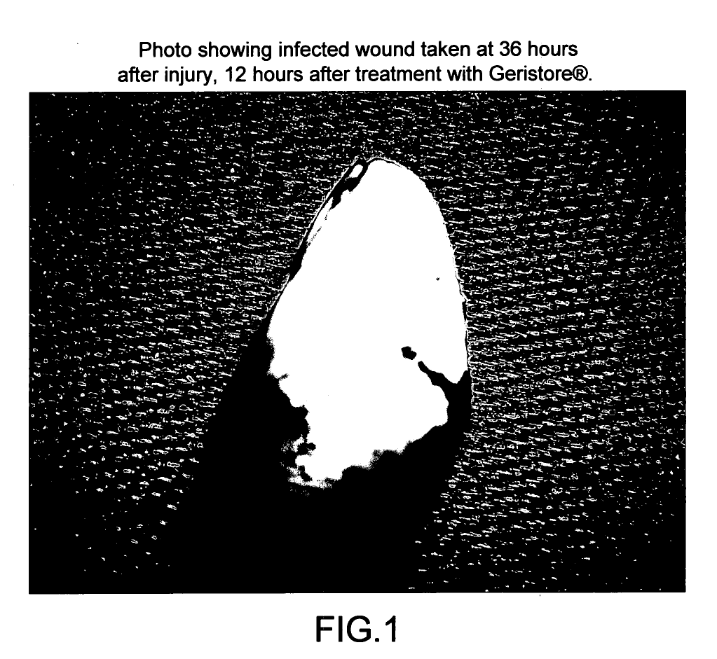

FIG. 2. Photo shows an infected wound 36 hours after injury, 12 hours after treatment with Geristore®.

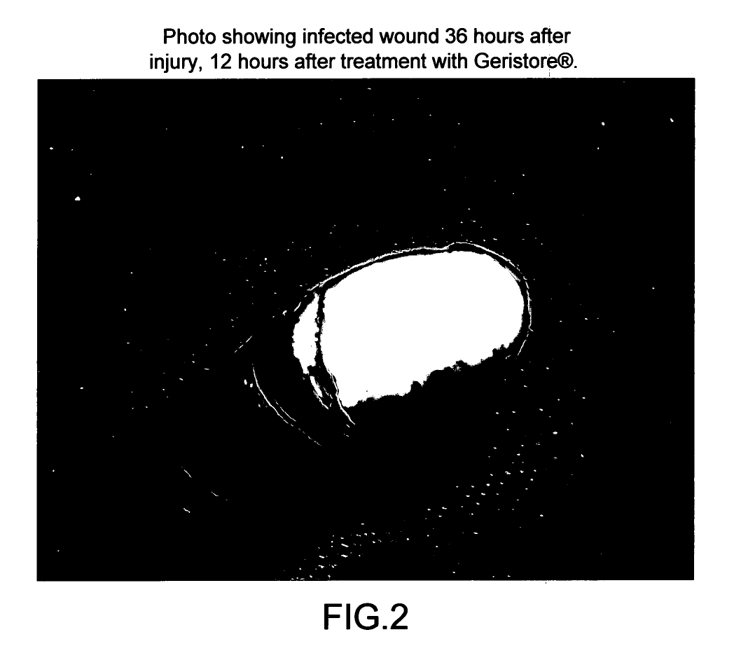

FIG. 3. Photo of infected wound taken 36 hours after injury, 12 hours after treatment with Geristore®.

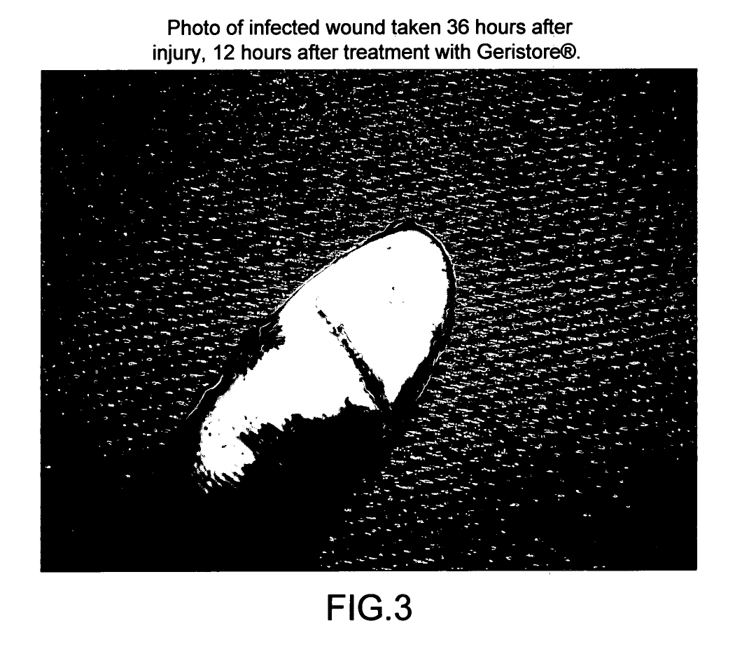

FIG. 4. Photo of wound 36 hours after treatment with Geristore®.

FIG. 5. Photo of wound 36 hours after treatment with Geristore®.

FIG. 6. Photo of wound 36 hours after treatment with Geristore®.

FIG. 7. Photo of wound 60 hours after treatment with Geristore®.

FIG. 8. Photo of wound 60 hours after treatment with Geristore®.

FIG. 9. Photo of wound 60 hours after treatment with Geristore®.

FIG. 10. Photo of wound 60 hours after treatment with Geristore®.

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implicated in other compositions and methods, and that any such variation would be within such modifications that do not part from the scope of the present invention. Before explaining the disclosed embodiments of the present invention in detail, it is to be understood that the invention is not limited in its application to the details of any particular embodiment shown, since of course the invention is capable of other embodiments. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

The present invention is provides a treatment method for enhancing the natural healing of a wound. The present invention is directed to methods of applying ionomer-resins to wounds. Additional therapeutic compositions that promote the wound healing process may be incorporated into the ionomer-resins of the instant invention. For example, the ionomer-resin may include the incorporation of antimicrobial compositions, including but not limited to antifungal compositions, antibacterial compositions, anti-viral compositions and antiparasitic compositions. Examples of antimicrobial compositions that can be used in the present invention include, but are not limited to, isoniazid, ethambutol, clofazimine, rifabutin, fluoroquinolones, pyrazinamide, streptomycin, ofloxacin, ganciclovir, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, erythromycin, ciprofloxacin, clindamycin, lincomycin, acyclovir, trifluorouridine, pentamidine, atovaquone, paromomycin, diclazaril, acyclovir, trifluorouridine, foscarnet, penicillin, gentamicin and sparfloxacin.

Resins in accordance with the instant invention are typically a crosslinked heat and/or light set resins that contain hygroscopic groups that attract water to the coating. When the crosslinking is not too extensive, the primary coating can absorb enough water that it can swell. The amount of water that the primary coating can absorb can be as high as 37 weight percent. However, the degree of crosslinking of the primary coating is typically high enough that water absorption (determined according to ADA Specificaton No. 27) will not exceed about 10 weight percent, preferably not exceeding about 7 weight percent. The backbone of the polymer providing the hygroscopic groups of the resin phase of the primary coating is typically aliphatic and may contain groups therein that enhance the hydrophilicity of the resin phase. Though the primary coating's resin can be made by a condensation reaction, such as by low temperature resin formation by the reaction of a blocked polyisocyanate with a polyol, the resin is typically the in situ reaction product of one or more of a polymerizable ethylenically unsaturated organic monomer containing groups that are attractive to water. Thus the resin may contain:

(a) an ethylenically unsaturated-functional monomer that contains a hygroscopic group. Typical of such groups are hydroxyl, amide, amine, aliphatic ether, amine, hydroxyalkyl amine, hydroxyalkyl amide, pyrrolidone, ureyl, and the like. A particularly desirable thermosetting resin is based on 2-hydroxyethyl methylmethacrylate (“HEMA”), 2-hydroxyethyl acrylate, 2,3-dihydroxypropyl methacrylate, acrylamide, methacrylamide, hydroxyalkyl acrylamide, hydroxyalkyl methacrylamide, and the like materials.

(b) A linear polycarboxylic acid or acid salt that contains a plurality of pendant carboxyl or carboxylic acid salt groups. Particularly preferred polycarboxylic acids are polyacrylic acid, polymaleic acid, polyitaconic acid, or a copolymer of acrylic acid, maleic acid, fumaric acid or itaconic acid with other ethylenically unsaturated monomers such as methyl acrylate, ethylacrylate, methylmethacrylate, vinyl acetate, vinylmethylether, styrene, α-methylstyrene, vinylcyclohexane, dimethylfumarate, ethylene, and the like.

(c) A desirable coupling agent is an acrylic-type monomer that possesses acrylic-type unsaturation and contains a surface bonding group possessing one or more of the following groups:

| | |
|---|---|
| i) | an alkylene polyether; |
| ii) | hydroxyl |
| iii) | carboxyl |
| iv) | carboxylic acid salt |
| v) | quaternary ammonium |
| vi) | tertiary amine |
| vii) | phosphoryl |
| viii) | phosphinyl |
| ix) | stannoyl |
| x) | amide |
| xi) | alkylene amine |

A preferred coupling agent is a simple aromatic substituted amino acid or its alkali metal salt such as the free acid or alkali metal salt of (i) N-phenylglycine, (ii) the adduct of N-(p-tolyl)glycine and glycidyl methacrylate.

(d) A number of resins rely on polyacrylyl substituted monomers to crosslink and chain extend the polymer that comes into existence on polymerization in the presence of an polymerization initiator. For example, the pure forms of hydroxyethylmethacrylate (“HEMA”) typically contain small amounts of ethylene glycol dimethacrylate which will crosslink a polymer based on HEMA. The degree of crosslink may be so minuscule as to have little effect on the ultimate properties of the polymer. Crosslinking agents are frequently added to HEMA based resins to impart a particular quality of crosslinking and toughness to the cured resin. For example, diethylene glycol dimethacrylate can otherwise lower the crosslink density of the resin which may impart toughness to the resulting cured polymer. Those types of crosslinkers would be considered a soft crosslinker, as defined above. However, in the practice of this invention, it is desired to use dual crosslinkers, one that is hard and one that is soft. In this respect, one may include the above crosslinker, in its normal impurity concentrations, as part of the soft crosslinker, but in the preferred embodiment, it is desirable to employ hard and soft crosslinkers that contain at least two acrylyl groups bonded to aromatic containing moiety(ies). The preferred hard crosslinking agent is one of (i) the esters or imides of pyromellitic acid dianhydride and 2-hydroxyethyl methacrylate or 2-aminoethyl methacrylate, or the corresponding acrylates, (ii) the ester or imides of 3,3′,4,4′-benzophenonetetracarboxylic dianhydride and 2-hydroxyethylmethacrylate or 2-aminoethyl methacrylate, or the corresponding acrylates, (iii) the esters and imide/amides of 4-trimellitic acid anhydride and 2-hydroxyethylmethacrylate or 2-aminoethyl methacrylate, or the corresponding acrylates, (iv) the ester or imides of 2,2-bis(3,4,-dianhydridophenyl)-1,1,1,3,3,3-hexafluoropropane and 2-hydroxyethyl methacrylate or 2-aminoethyl methacrylate, or the corresponding acrylates, and (iv) other compounds containing at least one group or moiety capable of free radical polymerization and at least one aromatic ring or moiety containing electron-withdrawing substituents that do not interfere with free radical polymerization. The soft crosslinker is typically an diacrylic or dimethacrylic ester or ether of bisphenol A, but also include as soft crosslinkers are the other glycol dimethacrylates and diacrylates mentioned herein. Preferred soft crosslinkers are ethoxylated bisphenol A dimethacrylate and the adduct of glycidylmethacrylate and bisphenol A.

(e) Fluoride may be included as a component in the resin. In the practice of the invention, the fluoride component will dissolve in water and to the extent the water is removed from the fluoride source, fluoride is carried with it. As noted above, the particularly desirable form of the fluoride component, is an inorganic fluoride in which the fluoride is present, e.g., in the form of an fluorosilicate structure or an alumina fluoride structure. The fluoride source of the patent is a glass composition in which the fluoride content is derived from an alkaline earth metal fluoride such as calcium fluoride, barium fluoride and strontium fluoride. A most preferred fluoride source is described in U.S. Pat. No. 5,360,770 which is incorporated herein by reference, particularly the examples and illustration of the patent that show how to make the fluoride source. As noted above, the resin is optionally provided with a leachable fluoride component. A particularly desirable form of the fluoride component, is an inorganic fluoride in which the fluoride is present, e.g., in the form of an fluorosilicate structure or an alumina fluoride structure.

(f) Also included in the formulation, as an optional ingredient, is a photoinitiator. According to one aspect this invention, the light-initiated curing of a polymerizable matrix material involves photosensitization of light-sensitive compounds by ultraviolet or visible light, which, in turn, initiates polymerization of the matrix material. The photoinitiator to be used in this invention comprises a combination of a photosensitive ketone and a tertiary amine. Typical photosensitive ketones include benzophenone, acetophenone, thioxanthen-9-one, 9-fluorenone, anthraquinone, 4′-methoxyacetophenone, diethoxyacetophenone, biacetyl, 2,3-pentadione, benzyl, 4,4′-methoxybenzil, 4,4′-oxidibenzil, and 2,3-bornadione (dl camphroquinone). Typical tertiary amines include ethyl-4-dimethyl amino benzoate, ethyl-2-dimethyl amino benzoate, 4,4′-bis(dimethylamino)benzophenone, N-methyldiethanolamine, and dimethylaminobenzaldehyde. A preferred combination of the photoinitiators is 2,3-bornanedione with ethyl-4-dimethyl amino benzoate. Other suitable initiator are illustrated in U.S. Pat. No. 4,674,980 to Ibsen, et al., the disclosure of which is incorporated by reference. Alternatively, any known photosensitizing system which can function effectively in a paste/paste composition when exposed to light may substitute for the above-named compounds or combinations. The amount of the photoinitiator should be sufficient to initiate polymerization in a selected resin and complete it in depth within about half a minute when the filler-resin composition is exposed to a visible-light output of at least 5,000 foot candles. In addition, any known free-radical scavenger (anti-oxidants) such as butylated hydroxytoluene can be used to scavenge small amounts of free radicals generated during extended shelf storage.

(g) The polymerization system of the composition may depend on effecting cure with either the photoinitiator or by use of a thermal initiator, which is a typical thermal curing agent known in the art. Illustrative of these are benzoyl peroxide, dicumyl peroxide, ditertiary butyl peroxide, tertiary butyl hydroperoxide, cumyl hydroperoxide, or other suitable peroxides may initiate polymerization of the polymerizable ethylenically unsaturated components of the primary coating. Addition of such thermal initiators is desirable to insure complete polymerization. Even when light alone does not cure the matrix material, the peroxide initiates curing of the uncured material thermally upon standing. Benzoyl peroxide may be used together with 2-hydroxyethyl-p-toluidine.

In formulating the resin, the selection of the ingredients in formulating the resin is narrowly critical. Illustrative of such a formulation is the paste/paste primary coating composition as set forth in Table 2.

TABLE 2

| | |
|---|---|
| Ingredients | Percentage by Weight |

Paste A

| | |
|---|---|
| Glass, fluoride source | 0-85 |
| Ethylenically unsaturated monomer, e.g., 2- | 3-40 |
| hydroxyethyl methacrylate | |
| Soft Crosslinker, e.g., Ethoxylated bisphenol A | 10-60 |
| dimethacrylate | |
| 2,3-bornanedione | 0.03-0.30 |
| Butylated hydroxytoluene | 0.001-1.0 |
| Benzoyl peroxide | 0.005-0.10 |
| Polycarboxylic acid, e.g., polyacrylic acid | 0-8 |
| Hard Crosslinker, e.g., PMDM | 2-20 |
| d-Tartaric acid | 0-1 |
| 2,2-Hydroxy-5-tert-octyl phenylbenzotriazole | 0.00-2 |
| Ethyl 4-dimethylaminobenzoate | 0.00-2 |

Paste B

| | |
|---|---|
| Glass, fluoride source | 0-70 |
| Ethylenically unsaturated monomer, e.g., 2- | 0-45 |
| hydroxyethyl methacrylate | |
| Soft Crosslinker, e.g., ethoxylated bisphenol A | 10-90 |
| dimethacrylate | |
| Coupling agent, e.g., Na NTG-GMA, NGT-GMA | 1-20 |
| Zinc oxide | 0-15 |
| Barium tungstate | 0-15 |
| Ethyl 4-dimethylamino benzoate | 0-2.0 |
| 2,3-bornanedione | 0.05-0.30 |
| Butylated hydroxytoluene | 0.005-0.10 |
| Titanium dioxide | 0.0-3.0 |
| 2,2-Hydroxy-5-tert-octyl phenylbenztriazole | 0.00-2 |

The two pastes, Paste A and Paste B, are preferably mixed well in equal amounts. The pastes may be mixed with a spatula or put onto a blade mixer prior to application to a wound. For example, the physician or technician may use the system by combining the pastes in the ratios desired, and then mixing them. The resulting paste is then applied directly to the wound as needed. The resin will self-cure in about 20-30 minutes, but cures instantly on exposure to light.

### EXAMPLE 1

### Preparation of Geristore®

In a small container add Part A of Geristore® (comprising approximately equal amounts of an aromatic dimethacrylate oligomer and 2-hydroxyethyl methacrylate along with small amount of benzoyl peroxide and a polymerization inhibitor). Mix in Part B of Geristore® (an aromatic dimethacrylate oligomer along with small amounts of a photoinitiator and a chelating agent). The Geristore® is now ready to be applied to the wound.

### EXAMPLE 2

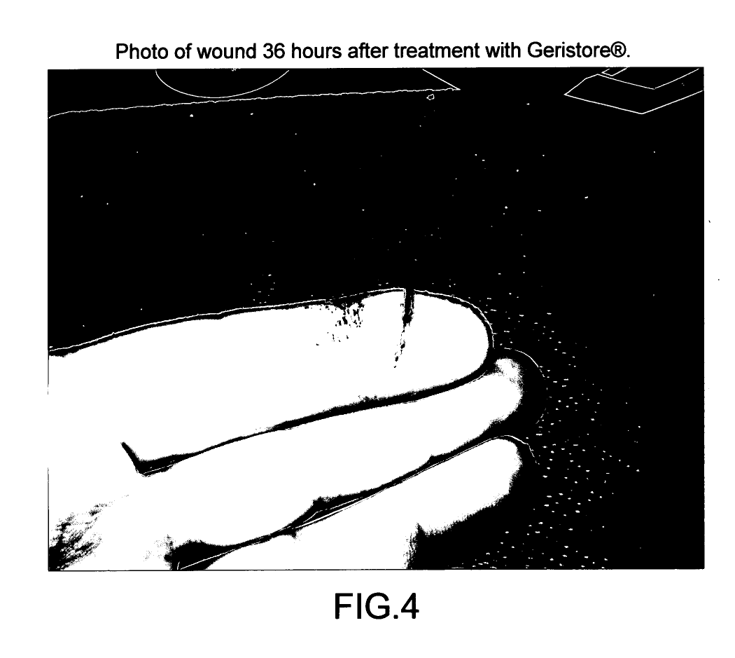

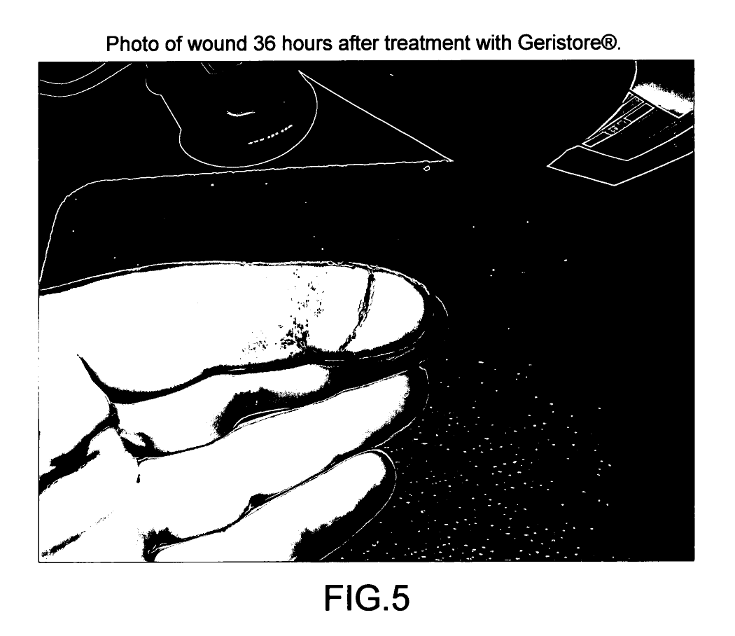

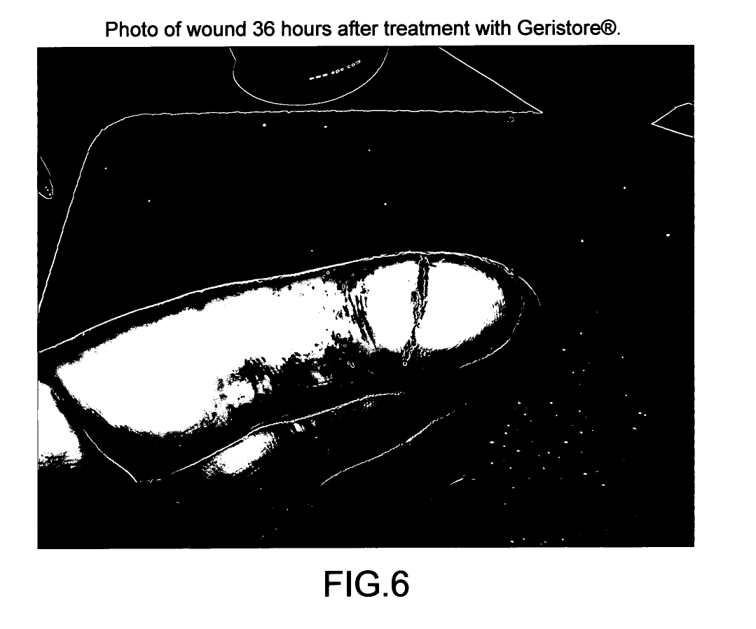

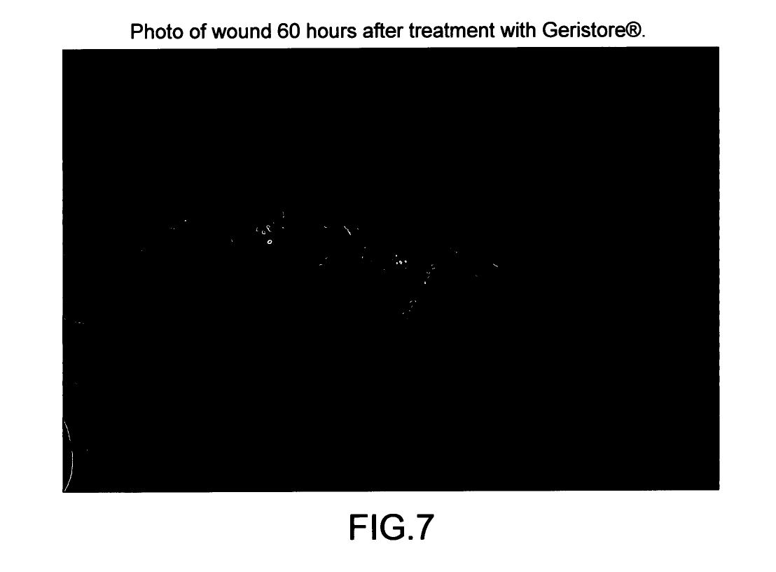

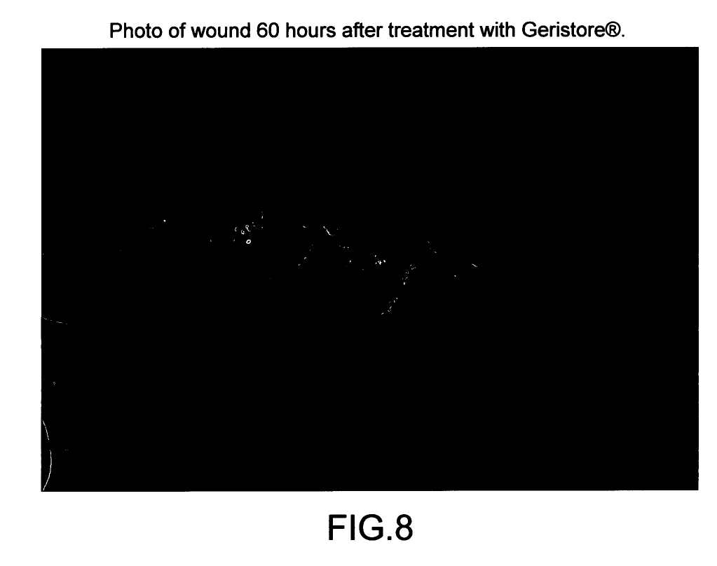

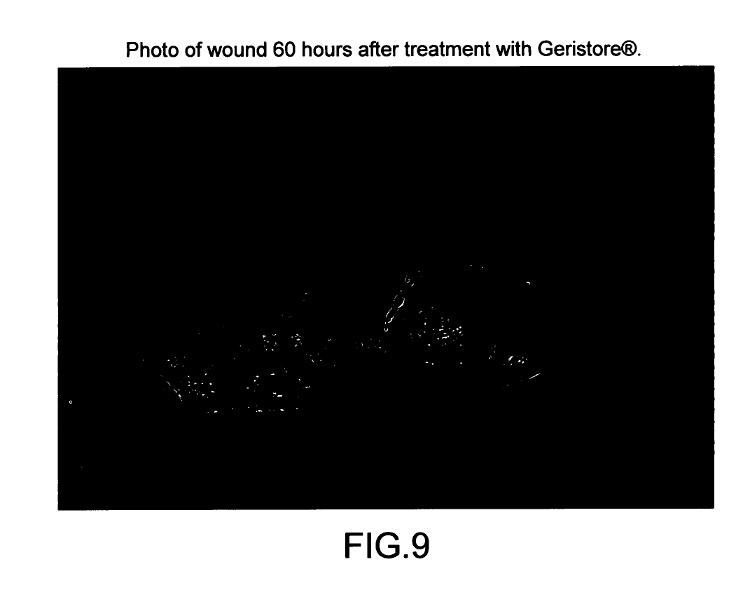

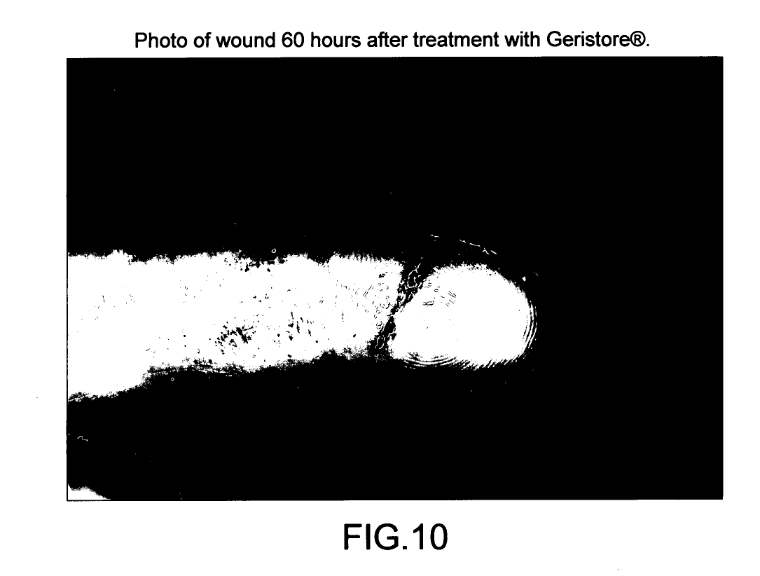

An individual was cut on his finger with a utility knife. The cut was approximately 7.5 mm in depth. The cut was not treated or disinfected in any way. The cut was bandaged for 24 hours with an untreated bandage. The bandage was removed after 24 hours at which time the cut was clearly infected and very little healing had occurred. The cut was then treated with Geristore®. Geristore® was applied directly to the wounded tissue, not as a covering or dressing. 12 hours after treatment with Geristore® the cut was visibly improved. See FIGS. 4-6. 36 hours after treatment the cut was infection free and was substantially healed. See FIGS. 7-10.

While the invention has been described with reference to certain exemplary embodiments thereof, those skilled in the art may make various modifications to the described embodiments of the invention without departing from the scope of the invention. The terms and descriptions used herein are set forth by way of illustration only and are not meant as limitations. In particular, although the present invention has been described by way of examples, a variety of compositions and methods would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A method of enhancing the natural healing of a wound comprising applying an uncured ionomer-resin to the surface of the wounded area.

2. The method of claim 1, wherein the ionomer-resin comprises fluoride.

3. The method of claim 1, wherein the ionomer-resin further comprises additional healing or antibiotic agents.

4. The method of claim 1, wherein the ionomer-resin further comprises an antimicrobial composition.

5. The method of claim 4, wherein the antimicrobial composition is selected from the group of isoniazid, ethambutol, clofazimine, rifabutin, fluoroquinolones, pyrazinamide, streptomycin, ofloxacin, ganciclovir, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, erythromycin, ciprofloxacin, clindamycin, lincomycin, acyclovir, trifluorouridine, pentamidine, atovaquone, paromomycin, diclazaril, acyclovir, trifluorouridine, foscamet, penicillin, gentamicin and sparfloxacin.

6. A method of treating a wound comprising:
(a) providing a compositions comprising an aromatic dimethacrylate oligomer, 2-hydroxyethyl methacrylate, benzoyl peroxide and a polymerization inhibitor;
(b) providing a composition comprising an aromatic dimethacrylate oligomer and a chelating agent;
(c) thoroughly mixing the compositions from step (a) and step (b) together to form a homogenous mixture; and
(h) applying the homogenous mixture to a wound.

7. The method of claim 6, further comprising the step of adding a pharmacological, or antibiotic composition to the mixture.

8. The method of claim 6, further comprising the step of adding an antimicrobial agent to the mixture.

9. The method of claim 8, wherein the antimicrobial composition is selected from the group of isoniazid, ethambutol, clofazimine, rifabutin, fluoroquinolones, pyrazinamide, streptomycin, ofloxacin, ganciclovir, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, erythromycin, ciprofloxacin, clindamycin, lincomycin, acyclovir, trifluorouridine, pentamidine, atovaquone, paromomycin, diclazaril, acyclovir, trifluorouridine, foscamet, penicillin, gentamicin and sparfloxacin.

10. The method of claim 6, further comprising the step of adding fluoride to the mixture.

11. A composition for enhancing wound healing comprising a hydrophilic water insoluble cross-linked resin.

12. The composition of claim 11, further comprising fluoride.

13. The composition claim 11, further comprising an antimicrobial composition.

14. The composition of claim 13, wherein the antimicrobial composition is selected from the group of isoniazid, ethambutol, clofazimine, rifabutin, fluoroquinolones, pyrazinamide, streptomycin, ofloxacin, ganciclovir, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, erythromycin, ciprofloxacin, clindamycin, lincomycin, acyclovir, trifluorouridine, pentamidine, atovaquone, paromomycin, diclazaril, acyclovir, trifluorouridine, foscamet, penicillin, gentamicin and sparfloxacin.
